(19) United States
(12) Patent Application Publication
(10) Pub. No.: US 2026/0021063 A1
(43) Pub. Date: Jan. 22, 2026

(54) COMPOSITIONS FOR FACILITATING PREVENTION OF SNORING AND SLEEP APNEA

(71) Applicant: Dennis Glaser, Gilbert, AZ (US)

(72) Inventor: Dennis Glaser, Gilbert, AZ (US)

(21) Appl. No.: 19/271,465

(22) Filed: Jul. 16, 2025

Related U.S. Application Data
(60) Provisional application No. 63/672,046, filed on Jul. 16, 2024.

(51) Int. Cl. A61K 31/19; A61K 9/00; A61K 9/48; A61K 31/405

(52) U.S. Cl. CPC A61K 31/19; A61K 9/0053; A61K 9/485; A61K 31/405

(57) ABSTRACT

The present invention provides a composition, a method of making the composition, and a method of using such composition for facilitating prevention of snoring and sleep apnea. The disclosed composition includes at least one active ingredient selected from Magnesium BHB, Calcium BHB, Sodium BHB, or 5-Hydroxytryptophan. According to an exemplary embodiment of the present invention, Silentium, which comprises one of the disclosed compositions, stops snoring and cures sleep apnea. The composition may be prepared by first adding and blending Magnesium BHB, then Calcium BHB, then Sodium BHB, and lastly, 5-Hydroxytryptophan. The composition may be taken orally in the form of a capsule or in the form of a solution.

### FIELD OF THE INVENTION

The present invention relates generally to drug, bio-affecting, and body treating compositions. More specifically, the present invention is compositions for facilitating prevention of snoring and sleep apnea.

### BACKGROUND OF THE INVENTION

The field of drug, bio-affecting, and body treating compositions is technologically important to several industries, business organizations, and/or individuals.

Existing remedies for preventing snoring and sleep apnea are deficient with regard to several aspects. Common sleep disorders such as snoring and sleep apnea affect millions worldwide, leading to disrupted sleep and impacting overall health. Current remedies often involve uncomfortable devices or invasive procedures, which many find unsatisfactory. Current remedies do not target the root causes of these conditions to provide a better quality of life for sufferers.

Therefore, there is a need for improved remedies for preventing snoring and sleep apnea that may overcome one or more of the above-mentioned problems and/or limitations.

### SUMMARY OF THE INVENTION

This summary introduces a selection of concepts in a simplified form, that are further described below in the Detailed Description. This summary is not intended to identify key features or essential features of the claimed subject matter. Nor is this summary intended to be used to limit the claimed subject matter's scope.

Disclosed herein is a composition for facilitating prevention of snoring and sleep apnea, in accordance with some embodiments. Further, Silentium Plus, an exemplary embodiment of the disclosed composition herein, stops snoring and cures sleep apnea. Further, each ingredient comprised in the composition targets different areas of your respiratory and muscular systems. Further, the disclosed composition may include at least one active ingredient comprising at least one of Magnesium beta-hydroxybutyrate (BHB), Calcium beta-hydroxybutyrate (BHB), Sodium beta-hydroxybutyrate (BHB), and 5-Hydroxytryptophan (5-HTP). Further, the composition may be prepared by first adding and blending the Magnesium BHB, then Calcium BHB, then Sodium BHB, and lastly, 5-Hydroxytryptophan. Further, the at least one active ingredient may be blended in that order at very low speeds. Further, the composition may be used as a weight loss agent mainly as a person may sleep so well that the person may have more energy the following day for burning calories.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 illustrates a Silentium Plus container for preventing snoring and sleep apnea, in accordance with some embodiments.

FIG. 2 is a flow chart of a method for preparing a composition for preventing snoring and sleep apnea, in accordance with some embodiments.

FIG. 3 is a flow chart of a method for using the composition for preventing snoring and sleep apnea, in accordance with some embodiments.

FIG. 4 illustrates the mixing container with the at least one active ingredient, in accordance with some embodiments.

FIG. 5 illustrates a tabular representation of the composition comprising the at least one active ingredient, in accordance with some embodiments.

### DETAIL DESCRIPTION OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein-as understood by the ordinary artisan based on the contextual use of such term-differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of compositions for facilitating prevention of snoring and sleep apnea, embodiments of the present disclosure are not limited to use only in this context.

The present disclosure describes compositions for facilitating prevention of snoring and sleep apnea. Further, Silentium, an exemplary embodiment of the disclosed composition herein, stops snoring and cures sleep apnea. Further, each ingredient comprised in the disclosed composition targets different areas of your respiratory and muscular systems. Further, the disclosed composition may include at least one active ingredient comprising at least one of Magnesium BHB, Calcium BHB, Sodium BHB, and 5-Hydroxytryptophan. Further, the composition may be prepared by first adding and blending the Magnesium BHB, then Calcium BHB, then Sodium BHB, and lastly, 5-Hydroxytryptophan. Further, the at least one active ingredient may be blended in that order at very low speeds. Further, the composition may be used as a weight loss agent mainly as a person may sleep so well that the person may have more energy the following day for burning calories.

Further, the disclosed composition may provide oral solutions for snoring and sleep apnea relief. Further, the composition may be designed to address the common discomforts experienced during sleep. Further, innovative medications comprised in the composition may offer a proven and effective approach to improving sleep quality and reducing snoring episodes.

Further, Silentium Plus, an exemplary embodiment of the disclosed system herein, may harness advanced scientific research and cutting-edge technology to deliver an unparalleled solution. Unlike other conventional snoring and sleep apnea remedies, the disclosed composition may be conveniently taken orally, eliminating the need for uncomfortable devices or invasive procedures.

Snoring and sleep apnea can have a significant impact on one's overall well-being, affecting not only the quality of sleep but also relationships and daytime productivity. With Silentium and SilentiumPlus, individuals may finally experience a restful night's sleep, waking up refreshed and revitalized.

The unique formula of Silentium and SilentiumPlus targets the root causes of snoring and sleep apnea, working to reduce airway blockages and enhance respiratory function. By providing relief from the frustrating symptoms associated with these conditions, these medications enable individuals and their loved ones to enjoy peaceful, uninterrupted sleep.

Silentium and SilentiumPlus have undergone rigorous clinical trials and demonstrated their efficacy in numerous studies, making them a trustworthy choice for those seeking snoring and sleep apnea relief. With their non-invasive nature and easy administration, the disclosed medications offer a safe and practical solution for individuals of all ages.

Say goodbye to restless nights and hello to a peaceful slumber with Silentium and SilentiumPlus. Further, the disclosed composition may help an individual to reclaim sleep and enhance overall well-being.

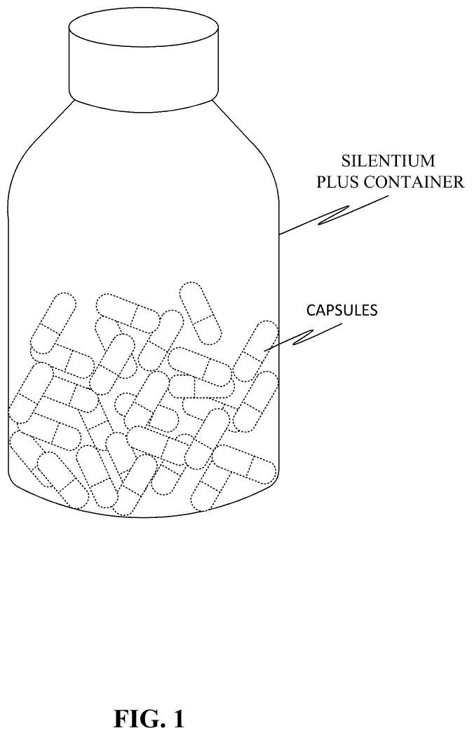

FIG. 1 illustrates a silentium plus container for preventing snoring and sleep apnea, in accordance with some embodiments. Accordingly, the silentium plus container may accommodate a plurality of capsules comprising the composition.

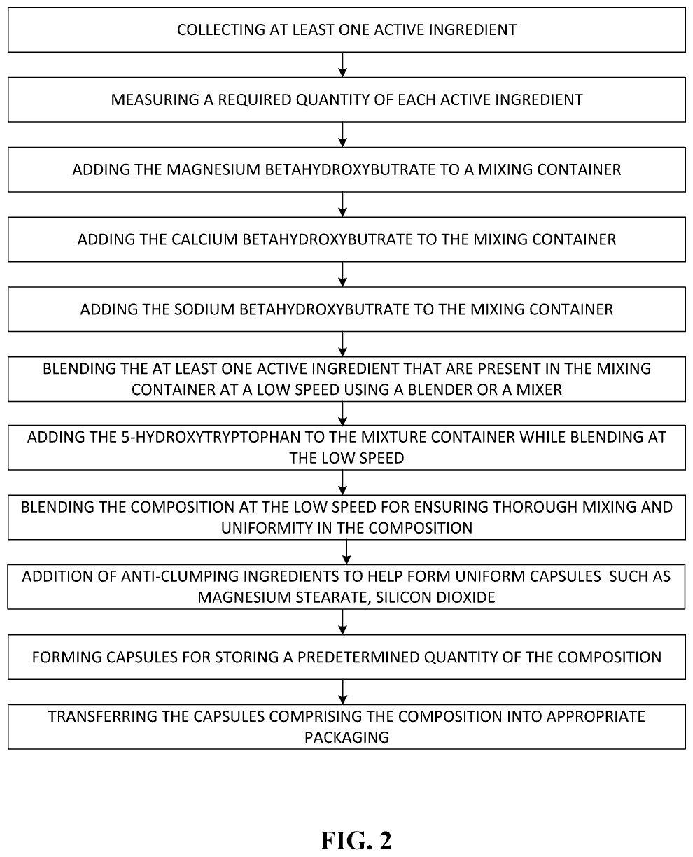

FIG. 2 is a flow chart of a method for preparing a composition for preventing snoring and sleep apnea, in accordance with some embodiments. Accordingly, the method may include collecting at least one active ingredient. Further, the at least one active ingredient may include Magnesium Beta-Hydroxybutyrate (BHB), Calcium Beta-Hydroxybutyrate (BHB), Sodium Beta-Hydroxybutyrate (BHB), and 5-Hydroxytryptophan.

Further, the method may include measuring a required quantity of each active ingredient. Further, each of the at least one active ingredient may be needed or required in a predetermined quantity (or the required quantity) to form the composition.

Further, the method may include adding the magnesium Hydroxybutyrate to a mixing container.

Further, the method may include adding the calcium Hydroxybutyrate to the mixing container.

Further, the method may include adding the sodium Hydroxybutyrate to the mixing container.

Further, the method may include blending the at least one active ingredient that are present in the mixing container at a low speed using a blender or a mixer. Further, the blending may ensure even distribution and avoid generating excessive heat.

Further, the method may include adding the 5-Hydroxytryptophan to the mixture container while blending at the low speed.

Further, the method may include blending the composition at the low speed for ensuring thorough mixing and uniformity in the composition.

Further, the method may include addition of anti-clumping ingredients to help form uniform capsules such as magnesium stearate and silicon dioxide.

Further, the method may include forming capsules for storing a predetermined quantity of the composition.

Further, the method may include transferring the capsules comprising the composition into appropriate packaging. Further, the packaging (e.g., bottles, pouches) may be sealed to prevent contamination. Further, the packaging may include the Silentium Plus container.

Further, in some embodiments, the method may include buffering the composition with the water and phosphate saline. Further, the method may include adding a mild analgesic to the mixing container.

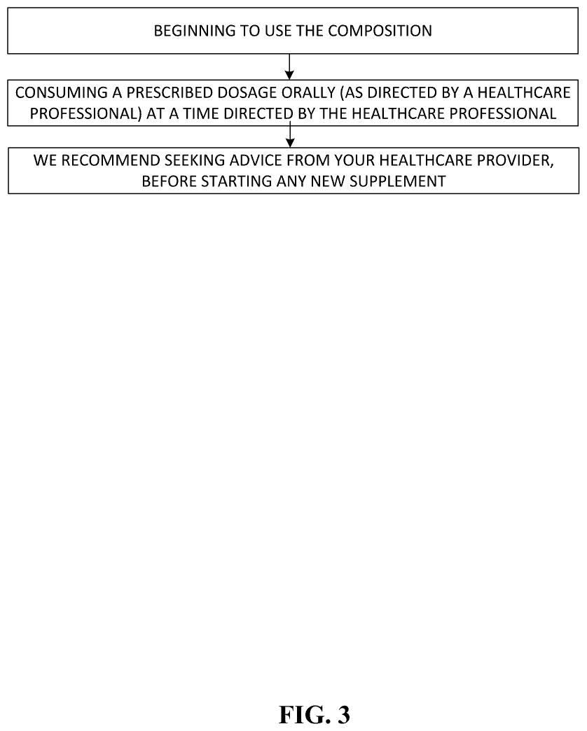

FIG. 3 is a flow chart of a method for using the composition for preventing snoring and sleep apnea, in accordance with some embodiments. Accordingly, the method may include a user beginning to use the composition. Further, the method may include the user consuming a prescribed dosage orally (as directed by a healthcare professional) at a time directed by the healthcare professional. Further, the method may include seeking an advice from a healthcare provider (or professional), before starting any new supplement. Further, the user may experience enhanced sleep quality and deep and restful sleep, allowing the user to wake up feeling refreshed and rejuvenated. Further, the user may experience a reduction in symptoms of sleep apnea, such as snoring and interrupted breathing, leading to a more peaceful sleep experience. Further, the user may experience healthy airflow and respiratory function as the Silentium optimizes oxygen intake during sleep, reducing sleep-related breathing issues.

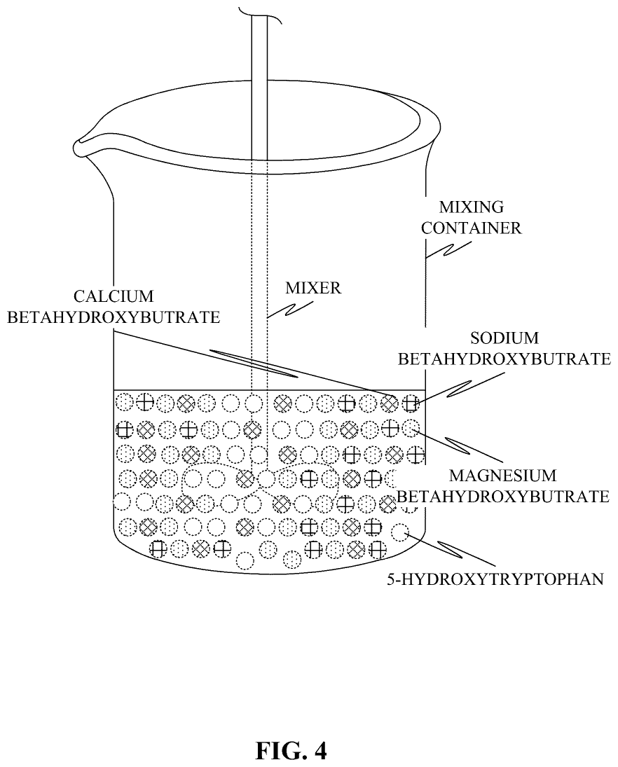

FIG. 4 illustrates the mixing container with the at least one active ingredient, in accordance with some embodiments.

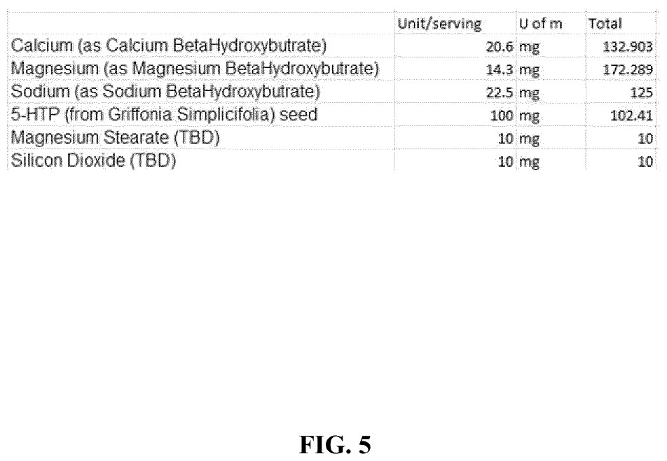

FIG. 5 illustrates a tabular representation of the composition comprising the at least one active ingredient, in accordance with some embodiments. Accordingly, the tabular representation shows a quantity or dosage of the at least one active ingredient.

As shown in FIG. 5, one preparation batch of the composition comprises 132.903 grams of calcium BHB, 172.289 grams of magnesium BHB, 125 grams of sodium BHB, 102.41 grams of 5-Hydroxytryptophan (5-HTP) (derived from Griffonia Simplicifolia seeds), 10 grams of magnesium stearate (TBD), and 10 grams of silicon dioxide. The final product is in the form of capsules. Each capsule comprises 20.6 mg calcium BHB, 14.3 mg magnesium BHB, 22.5 mg sodium BHB, and 100 mg 5-HTP.

Although the invention has been explained in relation to its preferred embodiment, it is to be understood that many other possible modifications and variations can be made without departing from the spirit and scope of the invention.

What is claimed is:

1. A composition for facilitating prevention of snoring and sleep apnea, the composition comprising:
at least one of the active ingredients selected from a group consisting of magnesium BHB, calcium BHB, sodium BHB, and 5-Hydroxytryptophan.

2. The composition of claim 1 further comprising an anti-clumping ingredient selected from a group including magnesium stearate and silicon dioxide.

3. The composition of claim 1 further comprising phosphate saline and water.

4. The composition of claim 1, wherein the composition is in the form of capsules.

5. The composition of claim 3, wherein the composition is in the form of an aqueous solution.

6. A method of facilitating prevention of snoring and sleep apnea for an individual in need thereof, the method including orally administering to the individual a composition comprising at least one of the active ingredients selected from a group consisting of magnesium BHB, calcium BHB, sodium BHB, and 5-Hydroxytryptophan.

7. The method of claim 6, wherein the composition further comprises an anti-clumping ingredient selected from a group including magnesium stearate and silicon dioxide.

8. The method of claim 6, wherein the composition further comprises phosphate saline and water.

9. The method of claim 6, wherein the composition is administered orally in the form of a capsule.

10. The method of claim 8, wherein the composition is administered orally in the form of an aqueous solution.

11. A method of making a composition for facilitating prevention of snoring and sleep apnea, the method comprising following steps:
Providing at least one of the active ingredients selected from a group consisting of magnesium BHB, calcium BHB, sodium BHB, and 5-Hydroxytryptophan;
Measuring a quantity of each of the active ingredients;
Adding the active ingredients in a mixing container by a sequential order of magnesium BHB, calcium BHB, sodium BHB, and 5-Hydroxytryptophan to form a mixture; and
Blending the mixture in the mixing container during and after the adding step hereabove at a slow speed so as not to producing excess heat during the blending until forming a uniform mixture.

12. The method of claim 11, wherein the step of blending the mixture is conducted by using a blender or a mixer.

13. The method of claim 11 further comprising a step of adding an anti-clumping ingredient to the mixture after adding the active ingredients, the anti-clumping ingredient selected from a group including magnesium stearate and silicon dioxide.

14. The method of claim 11 further comprising a step of consolidating the mixture into capsules.

15. The method of claim 11 further comprising a step of adding phosphate saline and water to form an aqueous solution.
